(19) United States
(12) Patent Application Publication
(10) Pub. No.: US 2022/0001060 A1
(43) Pub. Date: Jan. 6, 2022

(54) SMART UV DISINFECTION POD

(71) Applicant: Oren Aharon, Haifa (IL)

(72) Inventor: Oren Aharon, Haifa (IL)

(21) Appl. No.: 16/920,817

(22) Filed: Jul. 6, 2020

(51) Int. Cl. A61L 2/24; H04N 5/232; G06K 9/00; H04N 5/247; A61L 2/10

(52) U.S. Cl. CPC A61L 2/24; H04N 5/23299; G06K 9/00771; A61L 2202/14; A61L 2/10; A61L 2202/11; H04N 5/247

(57) ABSTRACT

Infected hospitals and habitats with antibiotic resistant pathogens are one of the most serious health threats. The lack of potent antibiotics and effective means against viruses creates a need for a solution which will disinfect the environment. This calls for an urgent alternative solution based on different technologies that eliminates the threat before the patient is infected. What is offered is a smart system based on AI technology that maps the most infected multi touch areas, and directs an ultraviolet beam of light to sterilize those surfaces. The light beam will eradicate microbial cells and viruses rapidly cleaning the suspected areas. A pan and tilt scanning of said light beam could be applied over the entire room. The purpose of this invention is to offer a camera-controlled multi-wavelengths UV beam delivery system, capable of disinfection of potential threats. Modes include pan and tilt full room disinfection and air sterilization mode.

### BACKGROUND OF THE INVENTION

This invention relates to an application of a disinfecting UV light beam, including blue light, antimicrobial ultraviolet irradiation and UVC to eradicate pathogenic germs and viruses.

Patient to patient infection is well known. However, evidence show that a contaminated environment is very significant for pathogen spread as well, especially in enclosed areas. High-touch surfaces are recognized as the first potential source of infectious contamination, secondly is infected air and floating droplets, and thirdly low-touch surfaces.

Traditional UV based disinfection systems for hospitals rely on delivering UV energy indiscriminately over a wide area. Typical systems include a high-power UV illuminator mounted on a moving carriage that scans an empty room distributing the UV power. This process is performed by special UV emitting lamps which radiate the environment with high energy, achieving eradication of pathogens by statistical illumination. This procedure is very time-consuming, expensive, and applied only once in a while, depending on the availability of said high power UV illuminator, which by itself is very expensive.

Technological advances as disclosed in this novel art are based on a video camera mapping the high touch and high suspected pathogenic areas and irradiating them with a directed beam when it is most convenient. The video camera, the directed light source and the computer are packaged together into a light pod featuring pan and tilt capability and preferably mounted on a high point. Unlike other systems, the proposed system has a built-in smart and to the point technology, irradiating the most contaminated surfaces first, and if possible (when there are no personnel or patients in the room) it will perform extensive room disinfection. Moreover, air disinfection could be performed regularly by exposing fanned air in an enclosed environment to UV radiation, preferably produced by said built-in directed light sources.

Periodically disinfecting high touch surfaces adjacent to patients will minimize contribution to secondary transmission.

There is a lot of interest in the development of effective and easily implemented disinfection strategies with “no touch” technologies, such as the use of the mobile UV-light disinfection system, which has the advantages of protecting the personnel from unnecessary exposure and has superior efficacy on eradication. Exposing the surfaces to high levels of UV radiation results in cellular damage of germs and viruses by photohydration, photo-splitting and photodimerization, thereby inhibiting cellular replication.

UVC can be generated from low-pressure mercury lamps that produce continuous UVC with a peak wavelength of 254 nm, and pulsed xenon lamps that emit pulsed light at high intensity, both in the spectrum of UVC (100-280 nm) and visible light (380-700 nm). Recent technological breakthrough culminated in the development of alternative sources of UV radiation by LED and lasers.

Using current mobile robots with UV and no personnel touch showed sustained reduction in surface microbial contamination, reduced cross contamination, and a reduced spread of multi-drug resistant bacterial infections.

The aim of this invention is to further increase the effectiveness of disinfection in contaminated areas using a combination of ultraviolet C (UVC) light-emitting devices with optical beam steering, and AI to perform a superior and accessible reduction of pathogen in the treated area.

The presented invention increases the effectiveness of disinfection in a matter which is free of drawbacks of current technologies.

### SUMMARY OF THE INVENTION

In disclosed art, we show a smart UV beam delivery system that can sterilize surfaces by aiming the highly disinfecting beam to preselected areas, preferably in empty rooms. The said preselected areas and locations are chosen by a special smart camera which continually records the high-touch surfaces and a built-in intelligent microprocessor will prioritize those surfaces according to their potential infection threat. The system is preferably composed by a pan/tilt device having a camera and a UVC light source mounted together and capable to observe and direct a light source to specific points in an enclosed environment. The camera will observe the environment and perform an analysis using artificial intelligence to define high-touch and other potentially infected areas. When convenient, a UV light beam will be directed to those surfaces and point to methodically clean and irradiate pathogens on said enclosed areas. For occupied rooms, the system will have a mode where it circulates the air in a cavity and expose it to high levels of UVC, thus sterilizing the air as well. Another possible embodiment is to have several pan/tilt mirrors at the ceiling level wherein the beam will be directed selectively to each mirror, and then the said mirrors will scan across the room from different directions to improve the quality of light disinfection.

The recent research “Evaluation of an Ultraviolet C (UVC) Light-Emitting Device for Disinfection of High Touch Surfaces in Hospital Critical Areas” shows that environmental cleaning and disinfection are important components on minimizing the healthcare person-to-person contamination. Studies have shown that the effectiveness of cleaning is limited and 5-30% of surfaces will remain contaminated, sometimes for a relatively long period. However, our technology which will be regularly applicated to the patient's room, has the potential to further reduce contamination since the main focus will be to decontaminate multi-touch surfaces first on a regular basis.

Moreover, the research shows that UV light disinfection has many advantages, and they prevent cellular replication. Although there are many sources of UV light, this patent will concentrate on LED & LD sources. The relevant wavelengths will be UVC 100-280 nm and UV 280-380, although any other wavelength which will be effective on viruses and germs irradiation is acceptable. To increase efficacy of eradication, sometimes best strategy will be to use several LEDs or LD working in parallel and illuminating at the same time the infected area. In case where wavelengths that may be dangerous to humans, the system will be equipped with motion sensors to prevent accidental exposure to UV radiation. One of the advantages of the system is that whenever there are surfaces that can be damaged by UV radiation, then the system will choose a different wavelength that is still effective but will not damage the plastic surfaces. The existing UV technology reduces cross-contamination and the spread of antibiotics-resistant—some of the studies show that from about 13% contamination UV cleaning reduced it to 0.4%. The disclosed technology has a potential of even better results when compared to other UV disinfections, due to the fact that the momentary exposure level is much higher since the beam is directed and focused on the surface to be disinfected.

The disclosed art introduces novel new technologies which will deliver multi-spectral UV beams directly to the affected areas and has the built-in capability to perform air disinfection as well.

The purpose of the present invention is to provide a multi-wavelengths UV disinfection system which is able to deliver high levels of UV light energy directly to contaminated surfaces. The system will automatically monitor and characterize the high-touch infected surfaces and will prioritize disinfection procedure. The said device will have several operational modes as follows:
Surfaces disinfection performed according to a procedure where the highest infected surface will be tackled first.
Air disinfection only mode.
System accidental radiation controlled by a built-in motion detector.
Special embodiments where radiation is delivered from several points around the suspected area.

### BRIEF DESCRIPTION OF DRAWINGS

Further advantages of the invention will emerge from the following descriptions and drawings, which are provided as non-limiting examples and in which:

FIG. 1 is a view of the pan/tilt pod according to the first embodiment of present invention.

FIG. 2 is a view of the pan/tilt pod in a second mode where it radiates an enclosed area to eradicate airborne viruses.

FIG. 3 is a view of the pan/tilt pod working in cooperation with multiple pan/tilt mirrors disposed across the room area.

FIG. 4 is a view of two said pan/tilt pods working in cooperation from two different directions.

FIG. 5 shows the beam's irradiations across multi-touch areas.

FIG. 6 shows in a schematic way the said pan/tilt mirror to be disposed across the room's area.

FIG. 7 shows in a schematic way the said pan/tilt camera with built-in germicidal UV illumination sources.

### DETAILED DESCRIPTION OF DRAWINGS

The basic configuration of disclosed system is comprised of a pan/tilt motorized camera with built-in UV illumination sources connected to the camera, which automatically illuminate in the direction of the camera's line of sight. Image processing will analyze the frequency of multi-touch areas in the room, will identify those areas and apply UV dosage accordingly by directing and swiping high power multi-wavelengths radiation to identified areas. The LED sources are equipped with collimating optics and attached to a motorized camera. The motorized camera is able to pan and tilt the system's line of sight across the observed room. When the room is occupied, the camera will be on a watch-only mode, identifying the multi-touch areas without violating the occupants' privacy. Moreover, this camera could be used for remote medical assistance when needed. To further increase its efficacy, a special layout of camera and mirrors is conceived where, for example but not limiting the application, a camera and three pan/tilt mirrors are mounted in the corner of the room, and are able to scan the UV radiation from different directions. The way this is performed is by directing the UV radiation from the camera towards one of the mirrors and then the pan/tilt mirror will disinfect the room from its point of view. By repeating the process with other mirrors, we can apply UV disinfection to the multi-touch surfaces from different directions, thus increasing its efficacy. A special mode of air-disinfection will be provided by creating a UV illuminated cavity wherein air is drawn in, sterilized and blown out without leaking UV radiation to the environment.

The UV disinfecting device and system is comprised of several sub-systems such as a pan/tilt camera having axes of rotation activated by motors to control the rotational movement of said camera and pointing it to a predeterminate location. A plurality of UV collimated LED or LD sources, are solidly attached to the pan/tilt camera, capable to deliver direct energy to predeterminate location. For location determination, an image processing device is used to analyze and observe the environment, defining the multi-touch areas within the camera's field of view. Furthermore, a microcontroller device controls the motors, driving the pan/tilt directions to deliver the disinfecting light energy to multi-touch areas. Moreover, image information could be streamed through the internet to a main controlling computer installed in the facility. For protection, a motion detector is used to detect motion and interrupt disinfection if motion is detected. The disinfection wavelengths used could be multiple wavelengths belonging to UVC, UVA, and UVB but can also include other wavelengths which will be later identified as germicidal. For increasing efficacy, multiple pan/tilt cameras, each equipped with multiple UV disinfecting source could be mounted in different locations, performing disinfection from several directions. A special configuration allows using one disinfection pan/tilt camera interconnected with multiple pan/tilt mirrors that can have a line of sight to the pan/tilt camera, receive the UV energy and redirect it to different locations across the room.

The present invention offers a method and device for active disinfection of enclosed areas with a smart and to the point technology.

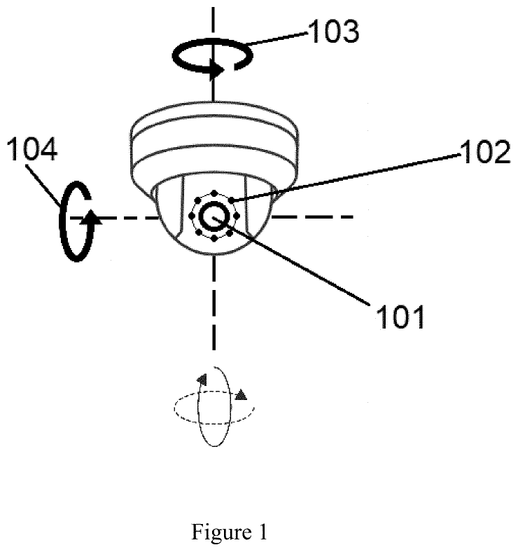

FIG. 1 describes the said pan/tilt pod, comprising of a video camera 101, surrounded by multiple UV LEDs 102 with a single wavelength or a combination of multiple wavelengths. The mechanics of the pod are designed in such a way that the line of sight of camera coincides with the illumination direction of LED devices. The pod has the capability to pan/tilt the said camera and LED devices according to external commands. Panning direction is denoted as 103 and the field direction is denoted as 104. The images are acquired by a computer system which will analyze the traffic in the room (computer system not shown), prioritize and identify the multi-touch surfaces.

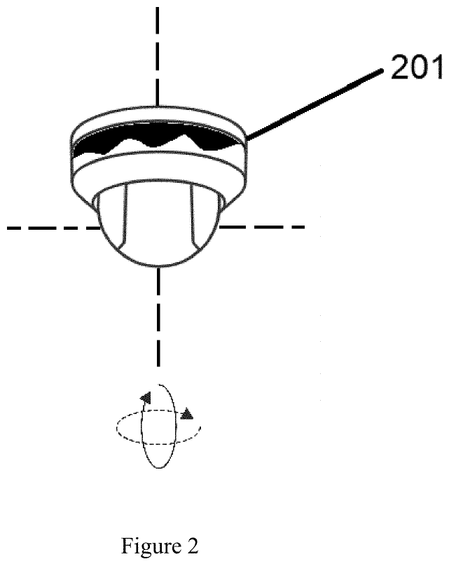

FIG. 2 describes the said pan/tilt pod in a second configuration wherein the camera and LEDs are pointed to an inner enclosed area denoted as 201, and illumination is directed to expose the air in said enclosed area to achieve air disinfection without external UVC light leakage. The air is constantly circulated in order to expose most of the room's air.

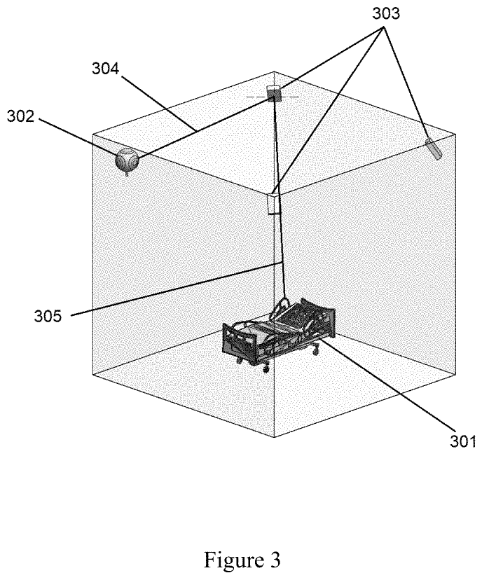

FIG. 3 describes a possible configuration in a patient's room environment wherein the bed denoted as 301 is on the room's floor, and the said pod denoted as 302 is in an upper position in respect with said bed. Several pan/tilt mirrors denoted as 303 are disposed in the upper part of the room in such a way that there is a line of sight between said pod and mirrors. Said pod can send the LED or LD UV illumination 304 towards one of the mirrors and the pan/tilt mirror can scan the UV light beam across the multi-touch surfaces present in the room. The scanning light beam, denoted as 305, will sterilize the multi-touch surfaces. Similarly, said pod can redirect the UV illumination toward a different mirror in the room to perform same function. By scanning from different directions, a better coverage of the multi-touch surfaces is achieved.

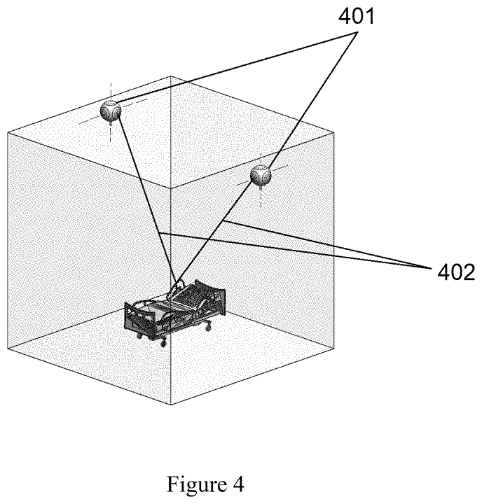

FIG. 4 describes a cooperation between two pods denoted as 401, and mounted on different positions at the upper part of the room to scan the multi-touch surfaces together or according to some predetermined program dictated by a micro-controller or a computing system, exposing the multi-touch areas to two or more light directions 402.

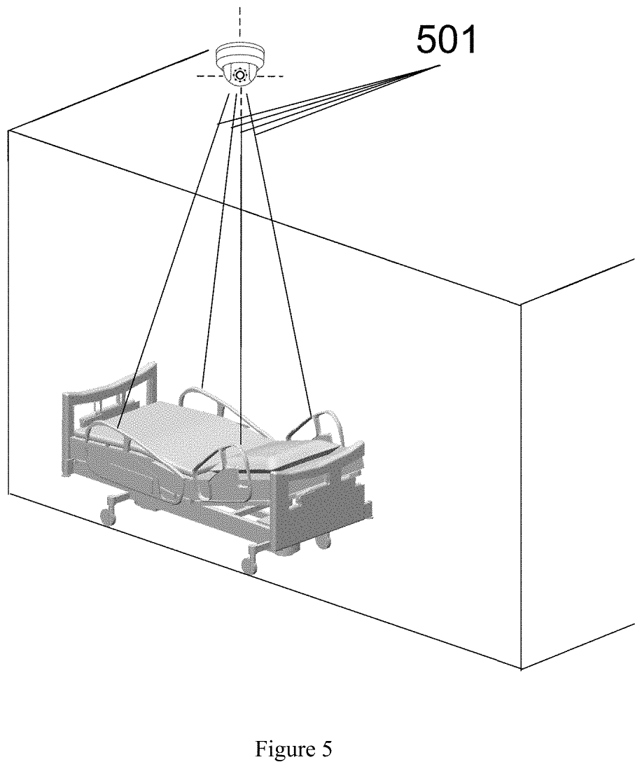

FIG. 5 describes a basic installation wherein one disinfection pod is located in the upper part of the room, and its illumination is projected to different positions. Those directions are denoted as 501.

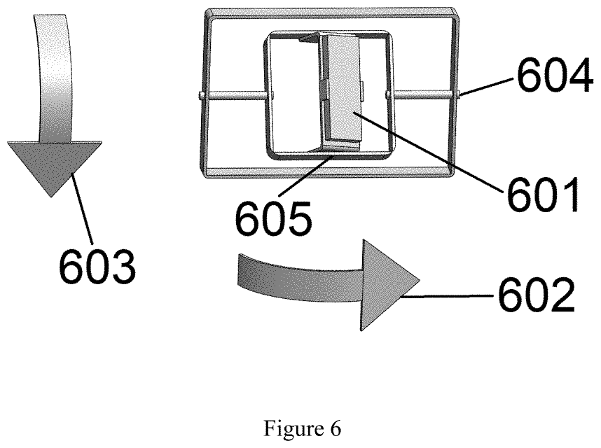

FIG. 6 describes the basic concept of a pan/tilt mirror which can receive a laser beam from said pod, and redirect it to any point in the room by using its pan/tilt capabilities. The mirror denoted as 601, can be panned in the direction denoted as 602 since it is mounted on an axis denotes as 605, which may be motorized. For tilting around the direction 603, the mirror is lowered around the axis denoted as 604.

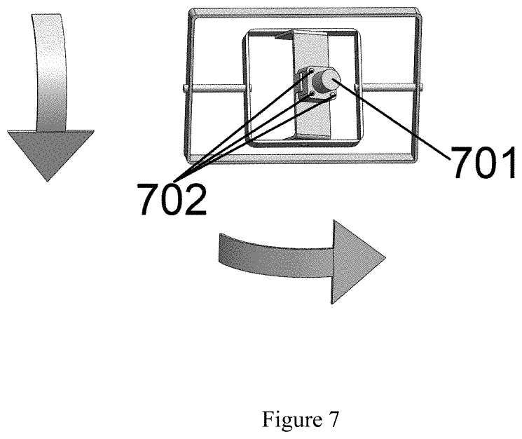

FIG. 7 describes the basic concept of the pan/tilt camera similar to the one described in FIG. 6, wherein the mirror was replaced by a camera denoted as 701, and has built-in laser illuminators at the appropriate wavelengths denoted as 702.

General Description

Although the detailed description contains many specifics for the purpose of illustration, a person of ordinary skill in the art will appreciate that many variations and alterations to the design details are within the scope of the invention. Very narrow and specific examples are used to illustrate particular embodiments; however, the invention described in the claims is not intended to be limited to only these examples, but rather includes the full scope of the attached claims. Accordingly, the following preferred embodiments of the invention are set forth without any loss of generality to, and without imposing limitations upon the claimed invention.

The above detailed description of the preferred embodiments is accompanied by drawings that form a part hereof, and in which specific embodiments are shown in an illustrative way. It is understood that other embodiments may be utilized and structural changes may be made without departing from the scope of the present invention.

1. A UV disinfecting device and system comprising of:
a pan/tilt camera having axes of rotation activated by motors to control the rotational movement of said camera and pointing it to a predeterminate location;
a plurality of UV collimated LED or LD sources, solidly attached to said pan/tilt camera, to deliver directed energy to said predeterminate location;
an image processing device to analyze the observed environment and define the multi-touch areas within the said camera's field of view; and
a microcontroller device to control the pan/tilt direction of said camera LED and deliver the UV light energy to multi-touch areas;

2. The device of claim 1 wherein processed image information is transferred through the internet to a main controlling computer.

3. The device of claim 1 having a motion detector to interrupt disinfection if the room is occupied.

4. The device of claim 1 wherein said plurality of UV collimated LED or LD sources have multiple wavelengths from UVC, UVA, UVB, or other germicidal wavelengths.

5. A UV disinfecting device comprising of:
Multiple pan/tilt cameras, each equipped with multiple UV disinfecting source mounted in different locations and performing disinfection from several directions.

6. A UV disinfecting device comprising of:
multiple pan/tilt mirrors, each having a line of sight to the pan/tilt camera, equipped with UV disinfecting sources, wherein the pan/tilt camera directs its line of sight to said mirrors, and UV beam direction is performed by the mirror device;
the said pan/tilt camera directs its line of sight to a different pan/tilt mirror, repeating the disinfection process from a different direction.
